(19) United States
(12) Plant Patent Application Publication
(10) Pub. No.: US 2016/0165783 P1
(43) Pub. Date: Jun. 9, 2016

(54) Capsicum annuum plant named 'SLP2B365'

(50) ;

(71) Applicant: Syngenta Participations AG, Basel (CH)

(72) Inventor: Binyamin Nir, Shikmim (IL)

(73) Assignee: Syngenta Participations AG, Basel (CH)

(21) Appl. No.: 14/998,721

(22) Filed: Feb. 5, 2016

Related U.S. Application Data
(63) Continuation of application No. 14/121,136, filed on Aug. 5, 2014.

(30) Foreign Application Priority Data
May 20, 2014 (NL) PPS1540

(51) Int. Cl. A01H 5/00

(52) U.S. Cl. PLT/258

(57) ABSTRACT

A new Capsicum annuum plant named ‘SLP2B365’ distinguished by its combination of seedless fruits, orange mature fruit color, and ability to set fruit under relatively hot conditions.

### CROSS-REFERENCE TO RELATED APPLICATIONS

This plant patent application is a continuation of co-pending U.S. patent application Ser. No. 14/121,136 filed Aug. 5, 2014, which claims the benefit under 35 U.S.C. §119(f) from Netherlands Plant Breeders' Rights application No. PPS1540, filed May 20, 2014, the disclosures of which are incorporated herein by reference in their entirety.

### LATIN NAME OF THE GENUS AND SPECIES OF THE PLANT CLAIMED

Capsicum annuum

### VARIETAL DENOMINATION

‘SLP2B365’

### BACKGROUND OF THE NEW PLANT

The present invention comprises a new and distinct cultivar of Capsicum annuum, designated ‘SLP2B365’.

The new C. annuum ‘SLP2B365’ was discovered and selected as a single plant in Gedera, Israel in 2011, from the progeny of a cross between SC09-F4-33-b as the female parent (not patented) and AJ06-F3-378-19 as the male parent (not patented).

The new cultivar was first asexually propagated by root cuttings in Bet Dagan, Israel in February, 2011 and has been asexually propagated since that time by cutting and rooting in Gedera, Israel and Almeria, Spain. The distinctive characteristics of this new C. annuum are stable and reproduce true to type through successive generations of asexual reproduction.

### SUMMARY OF THE INVENTION

“SLP2B365” is a distinctive variety of C. annuum characterized by the following traits, which have been repeatedly observed and in combination distinguish ‘SLP2B365’ as a new and distinct C. annuum cultivar:

1. Seedless fruits

2. Orange mature fruit color

3. Ability to set fruit under relatively hot conditions (e.g., 30° C. to 35° C. during the day and 25° C. to 30° C. at night).

Essentially all of the fruits of cultivar ‘SLP2B365’ are completely seedless. However, in cases where ‘SLP2B365’ has been grown in the vicinity of a fertile variety, there are occasional fruits with seeds due to cross-pollination by insects transferring pollen from the fertile variety to ‘SLP2B365’.

### BRIEF DESCRIPTION OF THE DRAWINGS

The accompanying photographs show typical characteristics of the new cultivar.

FIG. 1 shows mature fruits of cultivar ‘SLP2B365’.

### DETAILED BOTANICAL DESCRIPTION OF THE NEW CULTIVAR

The following is a detailed botanical description of a new and distinct variety of C. annuum known as ‘SLP2B365’. Those skilled in the art will appreciate that certain characteristics will vary with older or younger plants. Further, ‘SLP2B365’ has not been observed under all possible environmental conditions. Where dimensions, sizes, colors and other characteristics are given, it is to be understood that such characteristics are approximations or averages set forth as accurately as practicable. The variety may differ from the descriptions set forth herein with variations in environmental and cultural conditions.

‘SLP2B365’ is able to set fruit throughout the year when grown in a plastic greenhouse in the northern Negev region of Israel, including under cold (December to February) and hot (September) temperatures.

A technical description of the variety is provided below in Table 1 based upon observations of 5-month-old plants grown under greenhouse conditions in Gedera, Israel during the Fall-Winter season.

TABLE 1

| Characteristic | Value |
|---|---|
| Short technical description | Bush-type plant, medium inter - |
| | nodes, produces seedless fruit, |
| | good ability to set in relatively |
| Method of reproduction | hot conditions vegetatively |
| Plant: length of stem | propagated short-medium |
| Yes, Plant: shortened internodes | absent (indeterminant) |
| (in upper part) | |
| Plant: number of internodes between | none |
| 1st flower and shortened | |
| internodes (Varieties with | |
| shortened internodes only) | |
| Plant: length of internodes (on primary | medium |
| side shoots) (varieties without | |
| shortened internodes) | |
| Plant: anthocyanin coloration of nodes | present |
| Stem: Intensity of anthocyanin | medium-strong |
| coloration of nodes | |
| Stem: hairiness of nodes | weak |
| Plant: vigor | strong |
| Plant: height | medium |
| Leaf: length of blade | medium |
| Leaf: intensity of green colour | dark |
| Leaf: shape | ovate |
| Leaf: blistering | weak |
| Leaf: profile in cross section | flat |
| Leaf: glossiness | weak-medium |
| Leaf: Undulation of margin | light |
| Leaf: width of blade | medium |
| Flower: anthocyanin coloration | absent |
| in anther | |
| Peduncle: attitude | semi-drooping |
| Time of beginning of flowering (1st | early |
| flower on 2nd flowering node) | |
| Fruit: colour (before maturity) | green |
| Fruit: intensity of colour | medium |
| (before maturity) | |
| Fruit: anthocyanin coloration | absent |
| (before maturity) | |
| Fruit: general impression | conical |
| Fruit: attitude | drooping |
| Fruit: length | short-medium |
| Fruit: diameter | small |
| Fruit: ratio length-diameter | big |
| Fruit: shape in longitudinal section | triangular |
| Fruit: shape in cross section | circular |
| (at level of placenta) | |
| Fruit: sinuation of pericarp at basal part | very weak |
| Fruit: sinuation of pericarp | absent or very weak |
| excluding basal part | |
| Fruit: texture of surface | smooth or very slightly wrinkled |
| Fruit: colour (at maturity) | orange |
| Fruit: intensity of colour (at maturity) | medium-dark |
| Fruit: glossiness | medium |
| Fruit: stalk cavity | absent |
| Fruit: depth of stalk cavity | very shallow |
| Fruit: shape of apex (PBR) | very acute |
| Fruit: depth of interloculary grooves | very shallow |
| Fruit: number of locules | predominantly two |
| Fruit: length (cm) | 9 |
| Fruit: diameter (cm) | 2 |
| Fruit: thickness of flesh | thin |
| Stalk: length | Long |
| Stalk: thickness | Thin |
| Fruit: capsaicin in placenta | absent |
| Calyx: aspect | non enveloping |
| Seed | absent |
| Time of maturity | medium |
| Ripening can be compared with: | Kappy |
| Potato Virus Y pathotype 0 | susceptible |
| Potato Virus Y pathotype 1 | unknown |
| Potato Virus Y pathotype 2 | unknown |
| Tobamovirus pathotype P0 | highly resistant |
| Tobamovirus pathotype P0, P1 | susceptible |
| Tobamovirus pathotype P0, P1 P1-2 | susceptible |
| Tobamovirus pathotype P0, P1, P1-2, | susceptible |
| P1-2-3 | |
| Tomato Spotted Wilt Virus | susceptible |

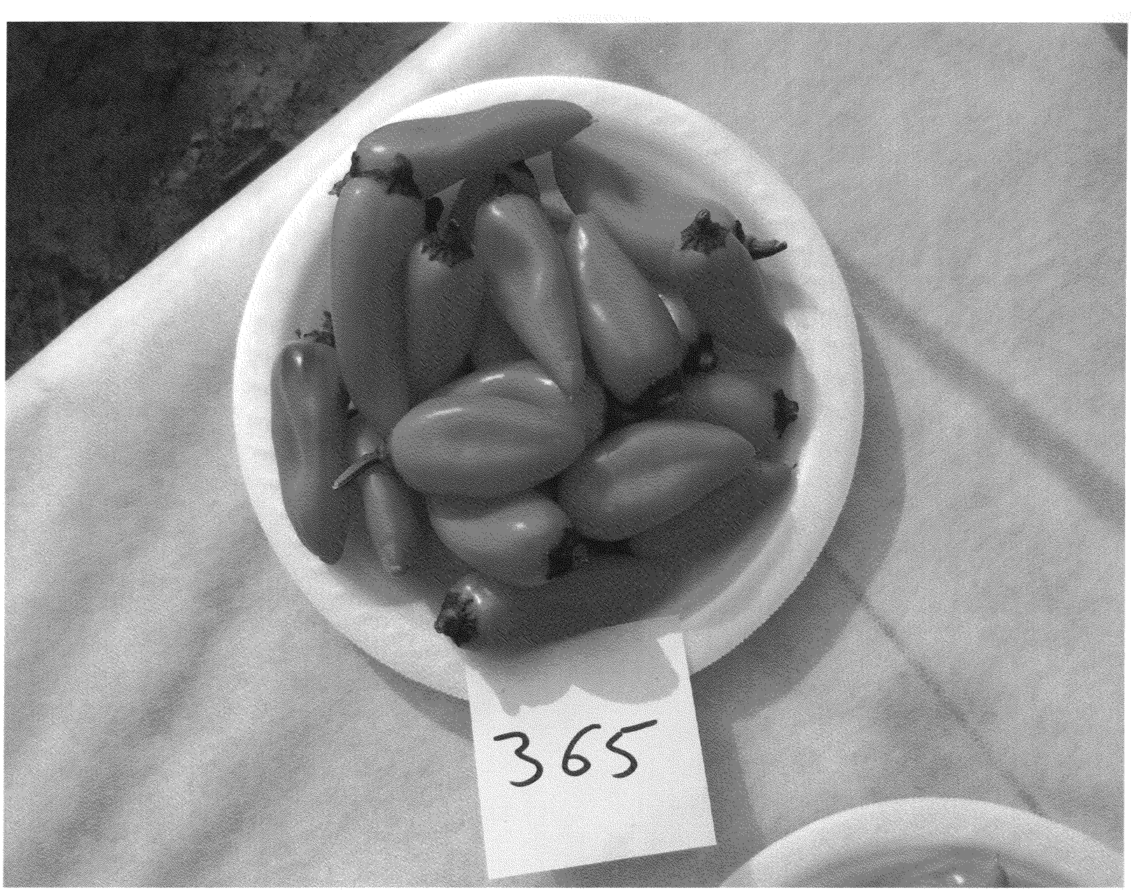

Further features and characteristics of cultivar ‘SLP2B365’ are apparent from FIG. 1.

Comparisons with other C. annuum cultivars.

‘SLP2B365’ is most similar to commercial C. annuum cultivar ‘SLP2B131’, which is also a seedless variety. As shown in Table 2, ‘SLP2B365’ can be distinguished from cultivar ‘SLP2B131’ at least with respect to mature fruit colour and season for fruit setting.

TABLE 2
SLP2B365 has in comparison with:

| | CHARACTERISTIC | | |
|---|---|---|---|
| DENOM- | IN WHICH THE | STATE OF | |
| INATION | COMPARISON | EXPRESSION | STATE OF |
| OF COM- | VARIETY IS | OF COM- | EXPRESSION |
| PARISON | DIFFERENT FROM | PARISON | OF |
| VARIETY | SLP2B365 | VARIETY | SLB2B365 |
| SLP2B131 | Fruit color at | Dark red | Orange |
| | maturity | | |
| SLP2B131 | Fruit setting season | Late (moderate | Early (Can set even |
| | | temperatures | in high |
| | | needed for good | temperatures) |
| | | set) | |

What is claimed is:

1. A new and distinct variety of Capsicum annuum plant named ‘SLP2B365’, substantially as described and illustrated herein.
